(19) United States
(12) Patent Application Publication
(10) Pub. No.: US 2023/0204500 A1
(43) Pub. Date: Jun. 29, 2023

(54) METHOD AND SYSTEM FOR DETECTING STAPHYLOCOCCUS AUREUS IN CHICKEN

(71) Applicant: Zhejiang University, Hangzhou (CN)

(72) Inventors: Yong HE, Hangzhou (CN); Ruicheng Qiu, Hangzhou (CN)

(73) Assignee: Zhejiang University, Hangzhou (CN)

(21) Appl. No.: 17/889,604

(22) Filed: Aug. 17, 2022

(30) Foreign Application Priority Data
Dec. 27, 2021 (CN) 202111610707.0

(51) Int. Cl. G01N 21/31; G16B 40/00; G06T 7/00; G06T 7/11; G06T 7/136; G06T 5/00; G01N 21/94

(52) U.S. Cl. CPC G01N 21/314; G16B 40/00; G06T 7/0004; G06T 7/11; G06T 7/136; G06T 5/002; G01N 21/94; G06T 2207/30128; G06T 2207/20081

(57) ABSTRACT

A method and system for detecting Staphylococcus aureus in chicken is provided. The method includes: obtaining hyperspectral images of samples; selecting spectral images at characteristic wavelengths based on the hyperspectral images of the samples, setting grayscale thresholds, and segmenting the selected spectral images to obtain a chicken sample region to be detected and a Staphylococcus aureus region to be detected; extracting hyperspectral data; extracting characteristic wavelengths after mixing the hyperspectral data of the chicken samples with the hyperspectral data of Staphylococcus aureus; selecting the hyperspectral data of the chicken samples corresponding to the extracted characteristic wavelengths to train a support vector machine model, thereby obtaining a detection model for Staphylococcus aureus; and detecting Staphylococcus aureus in chicken by using the detection model for Staphylococcus aureus.

### TECHNICAL FIELD

The present disclosure relates to the technical field of rapid nondestructive testing of food quality and safety, and in particular, to a method and system for detecting Staphylococcus aureus in chicken.

### BACKGROUND ART

Poultry meat products are prone to contamination by a variety of food-borne pathogenic bacteria during processing, causing quality degradation of the poultry meat products and giving rise to food safety problems. Staphylococcus aureus is a common pathogenic bacterium. A meat product contaminated by Staphylococcus aureus may cause a person who has eaten the meat product to have symptoms such as vomiting and diarrhea. Usually, biochemical identification means may be used to detect Staphylococcus aureus in foods. Chinese standard Microbiological examination of food hygiene—Examination of Staphylococcus aureus (GB4789.10-2016) provides a detection method under laboratory conditions. However, this method is complex to operate, poses high requirements on the professional skills of the detection personnel and the environmental conditions of the laboratory, and is destructive to samples. Moreover, the method may take a long detection time.

At present, it has been reported that such methods as enzyme-linked immunosorbent assay, immunofluorescence assay, and polymerase chain reaction have been used to detect food-borne pathogenic bacteria in foods, which, however, have the disadvantages of high costs, need for enrichment, long test cycles, etc. Many researchers have conducted food quality testing researches based on spectrum technologies, in which the spectrum technologies are employed to detect indicators such as spoilage bacteria, contaminants and pathogenic bacteria in meat products. Among them, terahertz and Raman spectrum technologies are widely used. However, when the terahertz or Raman spectrum technology is employed to detect pathogenic bacteria in meat products, the obtained detection signals may be weak, and it is usually necessary to design carriers of relevant materials for combination with samples to be detected. Pathogenic bacteria may change the constituents of meat products, leading to differences in spectral reflectance to visible light and near-infrared bands between a normal sample and a contaminated sample. It has been less reported on food-borne pathogenic bacteria detection based on hyperspectral data, especially on detection of Staphylococcus aureus in poultry meat products.

### SUMMARY

To address the problems of complex steps, low efficiency, great destructiveness and the like in detecting Staphylococcus aureus in poultry meat products, the present disclosure provides a method and system for detecting Staphylococcus aureus in chicken. The method and system can realize simple, rapid nondestructive detection of Staphylococcus aureus in chicken products and thus may meet the requirements of quality testing for chicken products during processing and sales.

The present disclosure provides the following solutions.

A method for detecting Staphylococcus aureus in chicken includes the following steps:

obtaining hyperspectral images of samples, the hyperspectral images of the samples including hyperspectral images of chicken samples and a hyperspectral image of Staphylococcus aureus, and the chicken samples including healthy chicken samples and contaminated chicken samples;

selecting spectral images at characteristic wavelengths based on the hyperspectral images of the samples, setting grayscale thresholds, and segmenting the selected spectral images to obtain a chicken sample region to be detected and a Staphylococcus aureus region to be detected;

extracting hyperspectral data of pixels in the chicken sample region to be detected and the Staphylococcus aureus region to be detected, respectively;

extracting characteristic wavelengths after mixing the hyperspectral data of the chicken samples with the hyperspectral data of Staphylococcus aureus;

selecting the hyperspectral data of the chicken samples corresponding to the extracted characteristic wavelengths to train a support vector machine model, thereby obtaining a detection model for Staphylococcus aureus; and

detecting Staphylococcus aureus in chicken by using the detection model for Staphylococcus aureus.

In some embodiments, the obtaining hyperspectral images of samples may specifically include:

inoculating Staphylococcus aureus in a Luria-Bertani (LB) agar medium for proliferation, and picking out typical colonies of proliferated Staphylococcus aureus for mixing with sterile distilled water to obtain Staphylococcus aureus solutions;

subjecting chicken breast slice samples to a sterilization operation under irradiation of an ultraviolet lamp and a contamination operation with the Staphylococcus aureus solutions at different concentrations, respectively, thereby obtaining the healthy chicken samples and the contaminated chicken samples; and

obtaining the hyperspectral images of Staphylococcus aureus in the LB agar medium, the healthy chicken samples and the contaminated chicken samples by using a hyperspectral imager in a vertical linear scanning manner.

In some embodiments, before the selecting spectral images at characteristic wavelengths based on the hyperspectral images of the samples, the method may further include:

performing a black-and-white correction on the hyperspectral images of the samples.

In some embodiments, the selecting spectral images at characteristic wavelengths based on the hyperspectral images of the samples, setting grayscale thresholds and segmenting the selected spectral images may specifically include:

selecting the hyperspectral image of Staphylococcus aureus at a wavelength of 648 nm as a grayscale image of Staphylococcus aureus, setting a grayscale threshold to 0.20, and segmenting the grayscale image of Staphylococcus aureus to obtain a binary image of Staphylococcus aureus;

selecting the hyperspectral image of the chicken sample at a wavelength of 622 nm as a grayscale image of the chicken sample, setting a grayscale threshold, segmenting the grayscale image of the chicken sample to obtain a binary image of the chicken sample; and

denoising the binary image of Staphylococcus aureus and the binary image of the chicken sample.

In some embodiments, before the mixing the hyperspectral data of the chicken samples with the hyperspectral data of Staphylococcus aureus, the method may further include:

smoothing the hyperspectral data of the chicken samples and the hyperspectral data of Staphylococcus aureus by using standard normal variate (SNV) and Savitzky-Golay (SG) algorithms.

In some embodiments, a competitive adaptive reweighted sampling (CARS) algorithm and a genetic algorithm (GA) are employed to extract the characteristic wavelengths, respectively.

The present disclosure further provides a system for detecting Staphylococcus aureus in chicken, including:

a sample hyperspectral image obtaining module configured to obtain hyperspectral images of samples, the hyperspectral images of the samples including hyperspectral images of chicken samples and a hyperspectral image of Staphylococcus aureus, and the chicken samples including healthy chicken samples and contaminated chicken samples;

a region-to-be-detected determining module configured to select spectral images at characteristic wavelengths based on the hyperspectral images of the samples, set grayscale thresholds, and segment the selected spectral images to obtain a chicken sample region to be detected and a Staphylococcus aureus region to be detected;

a hyperspectral data extracting module configured to extract hyperspectral data of pixels in the chicken sample region to be detected and the Staphylococcus aureus region to be detected, respectively;

a characteristic wavelength extracting module configured to extract characteristic wavelengths after mixing the hyperspectral data of the chicken samples with the hyperspectral data of Staphylococcus aureus;

a training module configured to select the hyperspectral data of the chicken samples corresponding to the extracted characteristic wavelengths to train a support vector machine model, thereby obtaining a detection model for Staphylococcus aureus; and

a detection module configured to detect Staphylococcus aureus in chicken by using the detection model for Staphylococcus aureus.

The present disclosure permits the use of the hyperspectral imaging technology in detecting Staphylococcus aureus in chicken, and can realize real-time, rapid nondestructive detection of pathogenic bacteria in chicken in combination with a machine learning recognition algorithm, thus providing an aid for food quality supervision.

### BRIEF DESCRIPTION OF THE DRAWINGS

To describe the technical solutions in embodiments of the present disclosure or in the prior art more clearly, the accompanying drawings required in the embodiments will be briefly described below. Apparently, the accompanying drawings in the following description show merely some embodiments of the present disclosure, and other drawings can be derived from the accompanying drawings by those of ordinary skills in the art without creative efforts.

FIG. 1 is a flowchart of a method for detecting Staphylococcus aureus in chicken according to an embodiment of the present disclosure.

FIG. 2 is a grayscale image of a Staphylococcus aureus sample at a characteristic wavelength.

FIG. 3 is a grayscale image of a chicken sample at a characteristic wavelength.

FIG. 4 is a binary image of Staphylococcus aureus resulting from segmentation.

FIG. 5 is a binary image of the chicken sample resulting from segmentation.

FIG. 6 is a diagram illustrating hyperspectral curves of Staphylococcus aureus, healthy chicken samples and contaminated chicken samples.

FIG. 7 is a diagram illustrating characteristic wavelengths extracted from hyperspectral data of Staphylococcus aureus, healthy chicken samples and contaminated chicken samples by using CARS and GA algorithms.

FIG. 8 is a diagram illustrating characteristic wavelengths extracted from hyperspectral data of healthy chicken samples and contaminated chicken samples by using CARS and GA algorithms.

### DETAILED DESCRIPTION OF THE EMBODIMENTS

The technical solutions in examples of the present disclosure will be described below clearly and completely with reference to the accompanying drawings in the examples of the present disclosure. Apparently, the described examples are merely some rather than all of the examples of the present disclosure. Based on the embodiments of the present disclosure, all other examples derived from the examples of the present disclosure by a person of ordinary skills in the art without creative efforts shall fall within the protection scope of the present disclosure.

An objective of the present disclosure is to provide a method and system for detecting Staphylococcus aureus in chicken. The method and system can realize simple, rapid nondestructive detection of Staphylococcus aureus in chicken products and thus may meet the requirements of quality testing for chicken products during processing and sales.

To make the objective, features and advantages of the present disclosure clearer and more comprehensible, the present disclosure will be further described in detail below in conjunction with the accompanying drawings and specific embodiments.

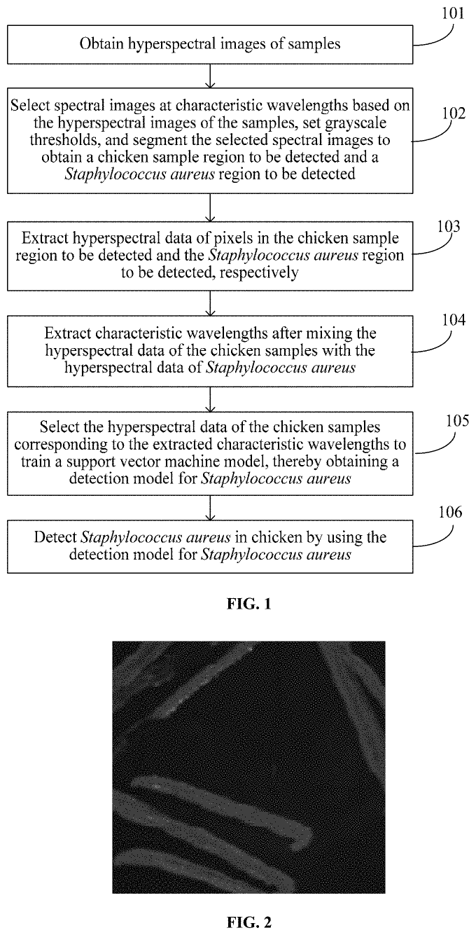

As shown in FIG. 1, a method for detecting Staphylococcus aureus in chicken provided in the present disclosure includes the following steps.

In step 101, hyperspectral images of samples are obtained. The hyperspectral images of the samples include hyperspectral images of chicken samples and a hyperspectral image of Staphylococcus aureus, and the chicken samples include healthy chicken samples and contaminated chicken samples.

In step 102, spectral images at characteristic wavelengths are selected based on the hyperspectral images of the samples, grayscale thresholds are set, and the selected spectral images are segmented to obtain a chicken sample region to be detected and a Staphylococcus aureus region to be detected.

In step 103, hyperspectral data of pixels in the chicken sample region to be detected and the Staphylococcus aureus region to be detected are extracted, respectively.

In step 104, the hyperspectral data of the chicken samples are mixed with the hyperspectral data of Staphylococcus aureus, and then characteristic wavelengths are extracted.

In step 105, the hyperspectral data of the chicken samples corresponding to the extracted characteristic wavelengths are selected to train a support vector machine model, thereby obtaining a detection model for Staphylococcus aureus.

In step 106, Staphylococcus aureus in chicken is detected by using the detection model for Staphylococcus aureus.

Step 101 specifically includes the following steps.

In step (1), purchased Staphylococcus aureus is inoculated in a Luria-Bertani (LB) agar medium for proliferation, and typical colonies from proliferated Staphylococcus aureus are picked up for mixing with sterile distilled water to obtain Staphylococcus aureus solutions.

In step (2), chicken breast is selected and segmented into slices having a thickness of about 0.5 cm, as samples to be detected. Some samples are subjected to disinfection and sterilization under irradiation of an ultraviolet lamp for 30 minutes to obtain healthy chicken samples, and the remaining samples are soaked in the Staphylococcus aureus solutions to obtain contaminated chicken samples.

In step (3), the hyperspectral images of Staphylococcus aureus in the LB agar medium, the healthy chicken samples and the contaminated chicken samples are obtained by using a hyperspectral imager in a vertical linear scanning manner.

In step (4), a black-and-white correction is performed on the hyperspectral images, regions of interest are set manually, and the hyperspectral images of chicken samples to be detected and Staphylococcus aureus are extracted.

Hyperspectral images from the hyperspectral imager under a white reference plate and a black reference plate are acquired, and the acquired hyperspectral images of the chicken samples and Staphylococcus aureus are corrected by using the following equation:

$${H}_{cal} = \frac{{H}_{raw} - {H}_{dark}}{{H}_{white} - {H}_{dark}}$$

where H_{cal} is a corrected hyperspectral image, while H_{raw} is an original hyperspectral image, H_{white} is a white reference plate image, and H_{dark} is a black reference plate image.

The Environment for Visualizing Images (ENVI) software is employed to manually set a rectangular box to extract the hyperspectral images of partial regions of the healthy chicken samples and the contaminated chicken samples.

Step 102 specifically includes the following steps.

The hyperspectral image of the Staphylococcus aureus samples at a wavelength of 648 nm is selected as a grayscale image of Staphylococcus aureus, the grayscale threshold is set to 0.20 to segment the image to obtain a binary image.

The hyperspectral image of the chicken samples at a wavelength of 622 nm is selected as a grayscale image of the chicken samples, the grayscale threshold is set by artificial statistic processing to segment the image so as to obtain a binary image.

And the obtained binary images are subjected to a denoising process.

Step 103 specifically includes: extracting the hyperspectral data of the obtained pixels, and smoothing the hyperspectral data using Standard Normal Variate (SNV) and Savitzky-Golay (SG) algorithms.

Step 104 specifically includes the following steps.

In step (1), a certain number of samples from the healthy chicken samples and the same number of samples from the contaminated chicken samples are picked out. Then a certain number of hyperspectral data are randomly selected from each sample, and a training set and a test set are generated by randomly assigning the certain number of hyperspectral data to respective sets in a ratio of 1:1.

In step (2), an equal number of hyperspectral data of Staphylococcus aureus are randomly selected according to a data size of the training set. Subsequently, the hyperspectral data of the healthy chicken samples and the contaminated chicken samples in the training set and Staphylococcus aureus are classified, and resultant categories are numbered, and the characteristic wavelengths are extracted by using CARS and GA algorithms.

Step 105 specifically includes the following steps.

In step (1), the hyperspectral data of the healthy chicken samples and the contaminated chicken samples in the training set at the characteristic wavelengths extracted by using the CARS and the GA algorithms are used as inputs to the SVM algorithm, to build the detection module for Staphylococcus aureus.

In step (2), the hyperspectral data of the chicken samples in the test sample is input to the detection model to obtain detection results. The detection results are compared with actual results, and the detection accuracy of the model is calculated according to the following equation, to estimate the detection performance of the model:

$$Accuracy ( \% ) = \frac{NT}{N} \times 100 ,$$

where NT represents the number of samples classified correctly, while N represents the total number of samples, and Accuracy represents the detection accuracy.

Specific examples are described below.

1. An LB agar medium was used to culture Staphylococcus aureus. Typical colonies were picked out from proliferated Staphylococcus aureus and mixed with sterile distilled water, to obtain Staphylococcus aureus solutions at respective concentrations of 3 log CFU/ml, 5 log CFU/ml and 6 log CFU/ml. Chicken breast was cut into slices, and some samples were sterilized under irradiation of an ultraviolet lamp to obtain healthy chicken samples, while some samples were soaked in the Staphylococcus aureus solutions to obtain contaminated chicken samples.

2. Original hyperspectral reflectance images of Staphylococcus aureus in the LB agar medium, the healthy chicken samples and the contaminated chicken samples were acquired within a waveband of 379 to 1023 nm by using a hyperspectral imager in a vertical linear scanning manner.

3. The acquired hyperspectral images were subjected to a black-and-white correction. ENVI software was employed to manually select and extract the hyperspectral images of regions of interest of the chicken samples, and background interference was removed.

4. The hyperspectral image of Staphylococcus aureus at the wavelength of 648 nm was extracted, as shown in FIG. 2. In this embodiment, with a pre-segmentation threshold manually set to 0.20, the image was segmented to generate a binary image, where white pixels were Staphylococcus aureus with a grayscale value of 1, and black pixels were background with a grayscale value of 0.

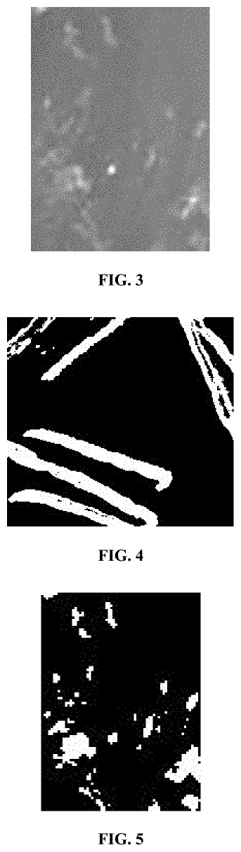

5. The hyperspectral images of the healthy and contaminated chicken samples at the wavelength of 622 nm were extracted, as shown in FIG. 3. In this embodiment, with a pre-segmentation threshold manually set to 0.55, the image was segmented to generate a binary image, where black pixels were the selected chicken region to be detected with a grayscale value of 0, and white pixels were a bright spot region of the chicken sample with a grayscale value of 1.

6. In this embodiment, a square structural element with a size of 2 pixels was selected to perform erosion operation on the binary image of Staphylococcus aureus and dilation operation on the binary image of the chicken sample, to remove small noise interference, with results being shown in FIG. 4 and FIG. 5, respectively. FIG. 4 shows the denoised image of Staphylococcus aureus, and FIG. 5 shows the denoised image of the chicken sample.

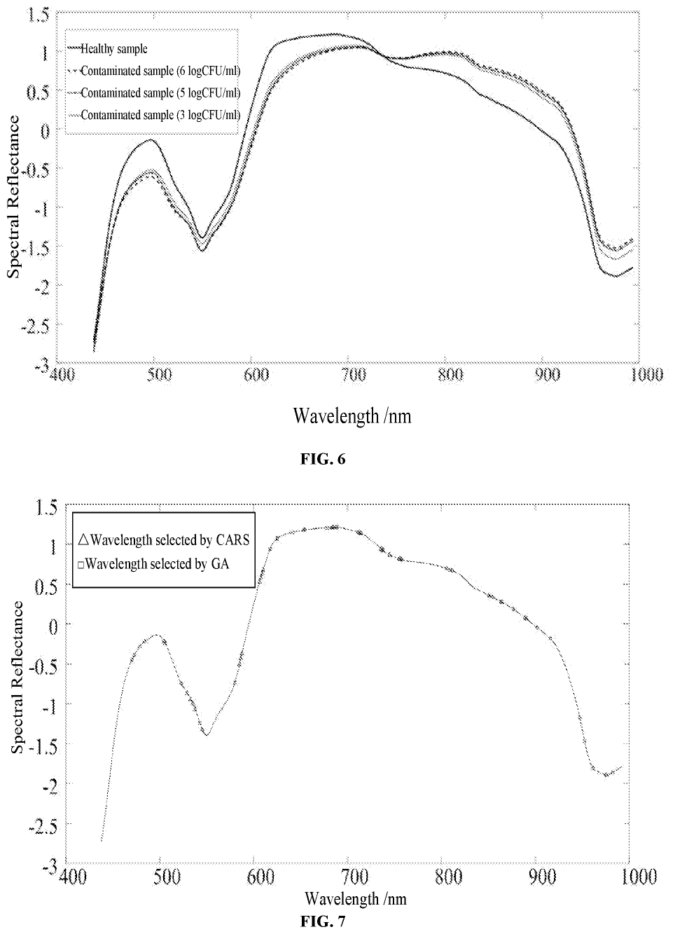

7. The hyperspectral data at pixels with the grayscale value of 1 were extracted from the binary image of Staphylococcus aureus, and the hyperspectral data of pixels with the grayscale value of 0 were extracted from the binary image of the chicken sample, and the hyperspectral data were smoothed by using SNV and SG algorithms. In this embodiment, the number of points of the SG algorithm was set to 15, and quadratic polynomial fitting was adopted. FIG. 6 shows smoothed spectral curves.

8. In this embodiment, 4 healthy chicken samples, and chicken samples contaminated at different concentrations (3 log CFU/ml, 5 log CFU/ml and 6 log CFU/ml, each corresponding to 4 samples) were selected randomly. The hyperspectral data at 1000 pixels were randomly selected from each sample, and divided into two groups in a ratio of 1:1. In this example, the ratio was set to 1:1, which is merely an instance and not limited thereto. The hyperspectral data of the healthy chicken samples and the hyperspectral data of the contaminated chicken samples were combined to finally generate a training set including the hyperspectral data of 2000 healthy chicken samples and 6000 contaminated chicken samples and a test set including the hyperspectral data of 2000 healthy chicken samples and 6000 contaminated chicken samples.

9. In this embodiment, 2000 samples were randomly selected from the obtained hyperspectral data of Staphylococcus aureus, and mixed with the samples of the generated training set. The data of Staphylococcus aureus, the healthy chicken samples and the chicken samples contaminated at three concentrations were divided into a first category, a second category, a third category, a fourth category, and a fifth category. The data were then processed by using the CARS and the GA algorithms to extract characteristic wavelengths, respectively. The CARS algorithm was set so that Monte Carlo sampling was performed for 400 times and the optimal solution was obtained when the root-mean-square error was minimum. The GA algorithm was set to have a population size of 60, crossover probability of 0.7, mutation probability of 0.01, and evolutional generation of 150. A wavelength that was selected for more than 20 times is taken as the characteristic wavelength. From selection results of the characteristic wavelengths shown in FIG. 7, 41 characteristic wavelengths were obtained by using the CARS algorithm: 478.95 nm, 483.81 nm, 504.55 nm, 505.78 nm, 529.12 nm, 532.82 nm, 535.29 nm, 537.76 nm, 545.18 nm, 584.99 nm, 586.24 nm, 587.49 nm, 606.29 nm, 607.54 nm, 608.80 nm, 610.06 nm, 652.96 nm, 654.22 nm, 683.43 nm, 684.70 nm, 711.49 nm, 712.76 nm, 714.04 nm, 735.80 nm, 737.08 nm, 738.56 nm, 744.77 nm, 755.03 nm, 756.32 nm, 757.60 nm, 811.65 nm, 850.37 nm, 851.66 nm, 854.24 nm, 864.57 nm, 889.13 nm, 890.42 nm, 947.26 nm, 961.46 nm, 975.65 nm, and 982.10 nm; and 17 characteristic wavelengths were obtained by using the GA algorithm: 470.46 nm, 472.88 nm, 522.97 nm, 542.71 nm, 580.00 nm, 617.60 nm, 625.16 nm, 641.57 nm, 678.34 nm, 688.52 nm, 805.21 nm, 809.07 nm, 863.28 nm, 876.20 nm, 902.05 nm, 916.26 nm, and 952.43 nm.

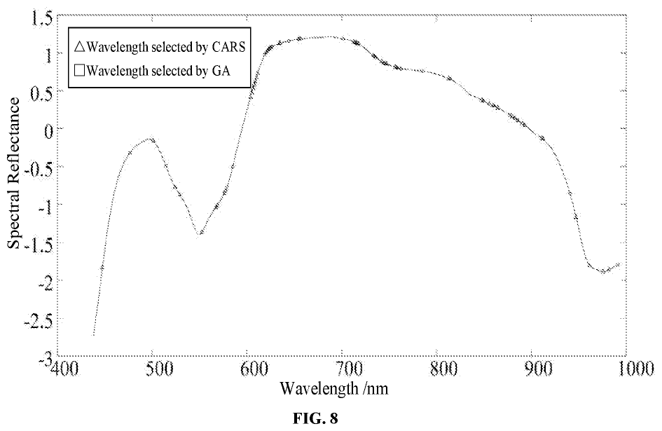

For comparison with the method of the present disclosure, only the samples in the generated training set were employed, and the data of the healthy chicken samples and the samples contaminated at three concentrations were divided into the first category, the second category, the third category, and the fourth category. The CARS and the GA algorithms were used to process the data to extract the characteristic wavelengths. The CARS and the GA algorithms were set as above, and in the GA algorithm, wavelengths that each were selected for more than 30 times were taken as the characteristic wavelengths. From selection results of the characteristic wavelengths shown in FIG. 8, 52 characteristic wavelengths were obtained by using the CARS algorithm: 447.54 nm, 500.88 nm, 524.20 nm, 552.62 nm, 567.53 nm, 568.77 nm, 603.78 nm, 605.03 nm, 606.29 nm, 607.54 nm, 608.80 nm, 610.06 nm, 611.31 nm, 633.99 nm, 635.25 nm, 654.22 nm, 655.49 nm, 656.76 nm, 712.76 nm, 714.04 nm, 715.32 nm, 716.60 nm, 717.88 nm, 733.23 nm, 734.51 nm, 735.80 nm, 742.20 nm, 744.77 nm, 746.05 nm, 747.33 nm, 756.12 nm, 757.60 nm, 758.89 nm, 812.94 nm, 814.23 nm, 847.78 nm, 849.07 nm, 855.83 nm, 859.41 nm, 860.70 nm, 864.57 nm, 880.08 nm, 882.66 nm, 886.54 nm, 890.42 nm, 893.00 nm, 911.09 nm, 912.39 nm, 947.26 nm, 961.46 nm, 975.65 nm, and 982.10 nm; and 22 characteristic wavelengths were obtained by using the GA algorithm: 476.52 nm, 514.36 nm, 529.12 nm, 576.25 nm, 577.50 nm, 578.75 nm, 584.99 nm, 618.86 nm, 620.12 nm, 621.38 nm, 622.64 nm, 623.90 nm, 625.16 nm, 626.42 nm, 644.10 nm, 701.27 nm, 762.74 nm, 784.60 nm, 877.49 nm, 885.25 nm, 940.81 nm, and 991.12 nm.

10. The hyperspectral data of the samples in the training set at the extracted characteristic wavelengths in FIG. 7 and FIG. 8 were extracted and input to a SVM model for training, and therefore, detection models for Staphylococcus aureus, namely model group 1 and model group 2, were obtained, respectively. In this embodiment, the SVM model was set as follows: a kernel function was set as a radial basis function; a penalty coefficient was set to 1.2, and the gamma function of the kernel function was set to 2.8. The generated test set was input to the obtained detection model for Staphylococcus aureus to obtain detection results, and detection results were compared with actual values. In the model group 1, the detection accuracy of the CARS and SVM based detection model for Staphylococcus aureus in chicken was 83.48%, and the detection accuracy of the GA algorithm and SVM based detection model for Staphylococcus aureus in chicken was 77.78%. In the model group 2, the detection accuracy of the CARS and SVM based detection model for Staphylococcus aureus in chicken was 84.30%, and the detection accuracy of the GA and SVM based detection model for Staphylococcus aureus in chicken was 77.24%.

The results indicated that rapid nondestructive detection of healthy chicken and chicken contaminated at different concentrations could be realized by using the hyperspectral data of Staphylococcus aureus, the healthy chicken samples and the contaminated chicken samples for extraction of the characteristic wavelengths and building the detection model for Staphylococcus aureus in chicken. Compared with the method of using the hyperspectral data of the healthy chicken samples and the contaminated chicken samples for extraction of the characteristic wavelengths, the method of the present disclosure could effectively reduce the number of the characteristic wavelengths but have substantially the same detection accuracy.

The present disclosure further provides a system for detecting Staphylococcus aureus in chicken, including:

a sample hyperspectral image obtaining module configured to obtain hyperspectral images of samples, the hyperspectral images of the samples including hyperspectral images of chicken samples and a hyperspectral image of Staphylococcus aureus, and the chicken samples including healthy chicken samples and contaminated chicken samples;

a region-to-be-detected determining module configured to select spectral images at characteristic wavelengths based on the hyperspectral images of the samples, set grayscale thresholds, and segment the selected spectral images to obtain a chicken sample region to be detected and a Staphylococcus aureus region to be detected;

a hyperspectral data extracting module configured to extract hyperspectral data of pixels in the chicken sample region to be detected and the Staphylococcus aureus region to be detected, respectively;

a characteristic wavelength extracting module configured to extract characteristic wavelengths after mixing the hyperspectral data of the chicken samples with the hyperspectral data of Staphylococcus aureus;

a training module configured to select the hyperspectral data of the chicken samples corresponding to the extracted characteristic wavelengths to train a support vector machine model, thereby obtaining a detection model for Staphylococcus aureus; and

a detection module configured to detect Staphylococcus aureus in chicken by using the detection model for Staphylococcus aureus.

The embodiments are described herein in a progressive manner. Each embodiment focuses on the difference from other embodiments, and the same and similar parts between the embodiments may refer to each other. Since the system disclosed in an embodiment corresponds to the method disclosed in another embodiment, the description is relatively simple, and reference can be made to the method description.

Specific examples are used herein to explain the principles and embodiments of the present disclosure. The foregoing description of the embodiments is merely intended to help understand the method of the present disclosure and its core ideas; besides, various modifications may be made by a person of ordinary skills in the art to the specific embodiments and the scope of application in accordance with the ideas of the present disclosure. In conclusion, the contents of the present description shall not be construed as limitations to the present disclosure.

What is claimed is:

1. A method for detecting Staphylococcus aureus in chicken, comprising:
obtaining hyperspectral images of samples, the hyperspectral images of the samples comprising hyperspectral images of chicken samples and a hyperspectral image of Staphylococcus aureus, and the chicken samples comprising healthy chicken samples and contaminated chicken samples;
selecting spectral images at characteristic wavelengths based on the hyperspectral images of the samples, setting grayscale thresholds, and segmenting the selected spectral images to obtain a chicken sample region to be detected and a Staphylococcus aureus region to be detected;
extracting hyperspectral data of pixels in the chicken sample region to be detected and the Staphylococcus aureus region to be detected, respectively;
extracting characteristic wavelengths after mixing the hyperspectral data of the chicken samples with the hyperspectral data of Staphylococcus aureus;
selecting the hyperspectral data of the chicken samples corresponding to the extracted characteristic wavelengths to train a support vector machine model, thereby obtaining a detection model for Staphylococcus aureus; and
detecting Staphylococcus aureus in chicken by using the detection model for Staphylococcus aureus.

2. The method for detecting Staphylococcus aureus in chicken according to claim 1, wherein the obtaining hyperspectral images of samples comprises:
inoculating Staphylococcus aureus in a Luria-Bertani (LB) agar medium for proliferation, and picking out typical colonies from proliferated Staphylococcus aureus for mixing with sterile distilled water to obtain Staphylococcus aureus solutions;
subjecting chicken breast slice samples to a sterilization operation under irradiation of an ultraviolet lamp and a contamination operation with the Staphylococcus aureus solutions at different concentrations, respectively, thereby obtaining the healthy chicken samples and the contaminated chicken samples; and
obtaining the hyperspectral images of Staphylococcus aureus in the LB agar medium, the healthy chicken samples and the contaminated chicken samples by using a hyperspectral imager in a vertical linear scanning manner.

3. The method for detecting Staphylococcus aureus in chicken according to claim 1, wherein before the selecting spectral images at characteristic wavelengths based on the hyperspectral images of the samples, the method further comprises:
performing a black-and-white correction on the hyperspectral images of the samples.

4. The method for detecting Staphylococcus aureus in chicken according to claim 1, wherein the selecting spectral images at characteristic wavelengths based on the hyperspectral images of the samples, setting grayscale thresholds and segmenting the selected spectral images comprise:
selecting the hyperspectral image of Staphylococcus aureus at a wavelength of 648 nm as a grayscale image of Staphylococcus aureus, setting a grayscale threshold to 0.20, and segmenting the grayscale image of Staphylococcus aureus to obtain a binary image of Staphylococcus aureus;
selecting the hyperspectral image of the chicken sample at a wavelength of 622 nm as a grayscale image of the chicken sample, setting a grayscale threshold, segmenting the grayscale image of the chicken sample to obtain a binary image of the chicken sample; and
denoising the binary image of Staphylococcus aureus and the binary image of the chicken sample.

5. The method for detecting Staphylococcus aureus in chicken according to claim 1, wherein before the mixing the hyperspectral data of the chicken samples with the hyperspectral data of Staphylococcus aureus, the method further comprises:
smoothing the hyperspectral data of the chicken samples and the hyperspectral data of Staphylococcus aureus by using standard normal variate (SNV) and Savitzky-Golay (SG) algorithms.

6. The method for detecting Staphylococcus aureus in chicken according to claim 1, wherein a competitive adaptive reweighted sampling (CARS) algorithm and a genetic algorithm (GA) are employed to extract the characteristic wavelengths, respectively.

7. A system for detecting Staphylococcus aureus in chicken, comprising:
a sample hyperspectral image obtaining module configured to obtain hyperspectral images of samples, the hyperspectral images of the samples comprising hyperspectral images of chicken samples and a hyperspectral image of Staphylococcus aureus, and the chicken samples comprising healthy chicken samples and contaminated chicken samples;
a region-to-be-detected determining module configured to select spectral images at characteristic wavelengths based on the hyperspectral images of the samples, set grayscale thresholds, and segment the selected spectral images to obtain a chicken sample region to be detected and a Staphylococcus aureus region to be detected;
a hyperspectral data extracting module configured to extract hyperspectral data of pixels in the chicken sample region to be detected and the Staphylococcus aureus region to be detected, respectively;
a characteristic wavelength extracting module configured to extract characteristic wavelengths after mixing the hyperspectral data of the chicken samples with the hyperspectral data of Staphylococcus aureus;
a training module configured to select the hyperspectral data of the chicken samples corresponding to the extracted characteristic wavelengths to train a support vector machine model, thereby obtaining a detection model for Staphylococcus aureus; and
a detection module configured to detect Staphylococcus aureus in chicken by using the detection model for Staphylococcus aureus.
